(19) United States
(12) Patent Application Publication
(10) Pub. No.: US 2018/0135068 A1
(43) Pub. Date: May 17, 2018

(54) SUPPRESSING MSH1 FUNCTION

(71) Applicant: Michael E Fromm, Lincoln, NE (US)

(72) Inventor: Michael E Fromm, Lincoln, NE (US)

(21) Appl. No.: 15/658,695

(22) Filed: Jul. 25, 2017

Related U.S. Application Data
(60) Provisional application No. 62/367,933, filed on Jul. 28, 2016.

(51) Int. Cl. C12N 15/82; C12N 15/11; C12N 15/10

(52) U.S. Cl. CPC C12N 15/8261; C12N 15/8205; C12N 15/8206; C12N 2800/80; C12N 15/102; C12N 2310/20; C12N 15/11

(57) ABSTRACT

The present invention provides methods of identifying and producing dominant negative MSH1 genes in plants and methods of using plants comprising dominant negative MSH1 genes for Msh1 suppression.

### CROSS-REFERENCE TO RELATED APPLICATIONS

None.

### INCORPORATION OF SEQUENCE LISTING

The sequence listing contained in the file named “DominantNegatives_ST25.txt”, which is 17,867 bytes in size (measured in operating system MS-Windows), contains 32 sequences, and is contemporaneously filed with this specification by electronic submission (using the United States Patent Office EFS-Web filing system) and is incorporated herein by reference in its entirety. The information recorded in computer readable form is identical to the written sequence listing submitted in the nonprovisional patent application Ser. No. 15/658,695 filed on Jul. 25, 2017. This computer readable submission of sequences includes no new matter and is the same as the paper version submitted previously.

### STATEMENT REGARDING FEDERALLY SPONSORED RESEARCH OR DEVELOPMENT

None.

### BACKGROUND OF INVENTION

Plant genes often occur in multigene families and/or can occur as multiple homeologous genes in polyploid species such as wheat or allotetraploid species such as soybeans or from ancient polyploid genomes that have diverged over time in species such as corn (Zea mays). Creating loss of function knockdowns or mutations in a function or trait encoded by multiple members of a gene family or multiple homeologous gene copies is a more difficult problem than for a function or trait encoded by a single gene. Mutations in several or all of the gene family members is one possible solution. Another solution is to create a dominant negative mutant capable of suppression of the function of wild type (non-mutant) proteins, most often in a protein complex containing the same (homomeric) or different (heteromeric) protein subunits.

MutS proteins and their eukaryotic homologs have examples of dominant negative mutants in E. coli, yeast, and humans, although dominant negative mutations in MutS homologs in plants are not known. Dominant negative mutants would be useful in certain applications for crop improvement that involve suppression of MutS homology 1 (Msh1) in plants.

Epigenetic Modifications and Traits. MSH1 suppression is useful for producing useful heritable epigenetic traits (U.S. Patent Application No. 20120284814, U.S. Provisional Application 61/863,267, U.S. Provisional Application 61/882,140, and U.S. Provisional Application 61/901,349, U.S. Provisional Application 61/930,602, U.S. Provisional Application 61/970,424, U.S. Provisional Application 61/980,096, U.S. Provisional 61/983,520, U.S. Provisional 62/000,756, and U.S. Provisional 62/031,692 for CRISPR related aspects, each of which is incorporated by reference in its entirety.

Considerable progress has been made in targeting proteins to specific DNA sequences in the genomes of live cells. Zinc fingers, TALENS, and CRISPR/CAS9 proteins or protein/RNA complexes are experimentally amenable to changes in their amino acid sequences or RNA targeting sequences to facilitate their binding to specific DNA sequences. Of these, the most convenient method to target a protein to a specific DNA sequence is with the CRISPR/CAS9 protein/RNA complex. CRISPR proteins are members of a large Cas3 class of helicases found in many prokaryotes, herein referred to as CRISPR or CRISPR/CAS9. CRISPR/CAS9 class of proteins bind either a single guide RNA or two annealed RNAs, that target specific DNA sequences through DNA/RNA complementary base pairing, facilitated by the CRISPR/CAS9 protein unwinding of the DNA. Multiple single guide RNAs (sgRNAs) can be used concurrently, with examples of two, three, four, five, six, or seven. Most designs utilize repeats of an intact sgRNA gene with its own Pol III U6 or U3 promoter. A S. pyogenes single guide RNA (sgRNA) has the following design: 20 nucleotide base-pairing region that is complementary or homologous to the target DNA sequence, a 42 nt Cas9 recognition hairpin structure, and a 40 nt S. pyogenes terminator with a 3′ hairpin followed by 4 or more U nt). The general sequence format is: 5′-N20 target-guide RNA. Transcription starts at the N1 position, or a processed transcript that has a 5′ end at the N1 position. Promoters transcribed by RNA Polymerase II can be used to produce sgRNAs due to processing by internal ribozymes at the 5′ and/or 3′ ends of the sgRNA.

The CRISPR/CAS9 system can be used for DNA cleavage at the targeted sequence, which is then repaired in vivo randomly with various DNA sequence changes or by homologous recombination if a template is also provided (for a recent review in plants, see Bortesi and Fischer, 2015, Biotechnology Advances 33(10): 41-52). Predictive software for useful sgRNA designs is available and progress on the mechanisms of CRISPR DNA recognition is proceeding.

Sequence specific DNA binding proteins such as zinc fingers, TALENS, and CRISPR proteins are useful in plants as well. Single guide RNAs are typically expressed from U6 or U3 promoters in plants, such as the wheat U6 promoter; the rice U3 promoter; the maize U3 promoter; or the Arabidopsis or rice U6 promoters. Ribozyme processing of transcripts from Pol II transcribed genes increases the flexibility of the system. Other gene editing methods such as TALENs, RNA/DNA oligonucleotides, zinc-fingers, homologous recombination and gene repair are known to those skilled in the art.

### SUMMARY

Methods for identifying plant MSH1 dominant negative (MSH1-DN) mutants or MSH1-DN mutations capable of suppressing the function of normal MSH1 in a heterozygous genetic state (MSH1-DN/MSH1) in either diploid or polyploid plants are provided herein. Methods for producing a MSH1-DN mutation in an endogenous plant Msh1 gene are also provided herein.

In certain embodiments a method of identifying a MSH1-DN mutation comprising producing candidate MSH1-DN mutations in an isolated Msh1 gene that is subsequently transformed into a plant; screening plants or their progeny comprising said candidate MSH1-DN mutations for loss of MSH1 function when containing both mutant and normal Msh1 genes; and identifying a MSH1-DN mutation that suppresses MSH1 function in a plant containing both mutant and normal Msh1 genes is provided herein.

In certain embodiments a method of producing a MSH1-DN mutation in a endogenous Msh1 gene in a plant comprising mutagenizing a population of plant cells, seeds, or pollen, and producing plants from the mutagenized plant cells, seeds, or pollen; screening said plants, or their progeny, to identify plants comprising a MSH1-DN mutation identified by the method above; and identifying a plant comprising a MSH1-DN mutation is provided herein.

In certain embodiments a method of producing a MSH1-DN mutation in a endogenous Msh1 gene in a plant comprising mutagenizing a population of plant cells, seeds, or pollen, and producing plants from the mutagenized plant cells, seeds, or pollen; screening said plants, or their progeny, to identify plants comprising a MSH1-DN mutation at an MSH1 equivalent amino acid position selected from the group consisting of positions of the MSH1-DN mutations in Table 2; and identifying a plant comprising a MSH1-DN mutation are also provided herein.

In certain embodiments of the aforementioned method the MSH1-DN mutation position is selected from the group of MSH1-DN mutation positions in Table 2 is provided herein.

In certain embodiments a plant or crop plant comprising a MSH1-DN mutation selected from the group of MSH1-DN mutations in Table 2 is provided herein.

In certain embodiments a method for producing a plant exhibiting improved yield comprising the steps of: suppressing expression of endogenous MSH1 gene(s) in a plant or plant cell to obtain a first parental plant, wherein suppression of MSH1 expression is effected with a MSH1-DN; selfing the first parental plant or crossing the first parental plant to a second parental plant; recovering progeny plants or a progeny plant line from the self or cross of the first parental plant wherein MSH1 function is restored; and, selecting a progeny plant or a progeny plant line from a recovered progeny plant or plant line or from a self or outcross of a recovered progeny plant or plant line wherein the selected progeny plant or plant line exhibits improved yield in comparison to a control plant, wherein said improved yield is associated with one or more epigenetic changes in the nucleus of the progeny plant cells relative to the corresponding parental chromosomal loci and is heritable is provided herein. In certain embodiments of the aforementioned method the MSH1-DN mutation is at an MSH1 equivalent amino acid position selected from the group consisting of non-naturally occurring amino acids at MSH1 equivalent amino acid positions 768, 772, 773, 775, 882, 823, and 853. In certain embodiments of the aforementioned method the MSH1-DN mutation is at MSH1 equivalent amino acid position and is selected from the group of MSH1-DN mutations in Table 2 is also provided herein.

In certain embodiments a plant or crop plant, or progeny thereof, produced by any of the aforementioned methods is provided herein.

In certain embodiments of the aforementioned method, the crop plant is a member selected from the group of plants consisting of corn, soybean, cotton, grapes, wheat, rice, tomato, tobacco, millet, potato, sorghum, alfalfa, sunflower, canola, peanut, canola (Brassica napus, Brassica rapa ssp.), coffee (Coffea spp.), coconut (Cocos nucijra), pineapple (Ananas comosus), citrus trees (Citrus spp.), cocoa (Theobroma cacao), poplar, sugar beets (Beta vulgaris), sugarcane (Saccharum spp.), oats, barley, vegetables, ornamentals, and conifers.

In certain embodiments progeny of a plant or crop plant comprising a dominant negative MSH1 gene are provided herein. In certain embodiments a MSH1-DN mutation is within the endogenous chromosomal Msh1 gene. In certain embodiments a MSH1-DN is obtain by mutagenesis of a plant or plant cell or plant gamete and screening of the resulting plant cells or plants. In certain embodiments a MSH1-DN is obtain by gene-editing of a plant or plant cell or plant gamete and screening of the resulting plant cells or plants. In certain embodiments a MSH1-DN is identified by screening mutations in a Msh1 transgene expressed in plants, in certain embodiments a MSH1-DN mutation identified by screening mutations in a Msh1 transgene expressed in plants is subsequently identified in an endogenous Msh1 gene by mutagenesis or gene-editing in a plant cell, plant, or gamete.

### BRIEF DESCRIPTION OF THE DRAWINGS

FIG. 1. Clustal Omega protein domain alignments of the MSH1 ATPase domain of selected plant species, with three homeologs shown for wheat. The amino acid numbering scheme is shown below the amino acid alignments where the star in bold corresponds to the first digit of the three digit number below it, e.g., the first start from the top corresponds to position 768. SEQ ID NOs for FIGURE: Wheat_2AL, SEQ ID NO:1; Wheat_2DL, SEQ NO:2; Wheat_2BL, SEQ ID NO:3; Sorghum, SEQ ID NO:4; Zea_mays, SEQ ID NO:5; Glycine max_max, SEQ ID NO:6; Arabidopsis_thaliana, SEQ ID NO:7.

### DESCRIPTION

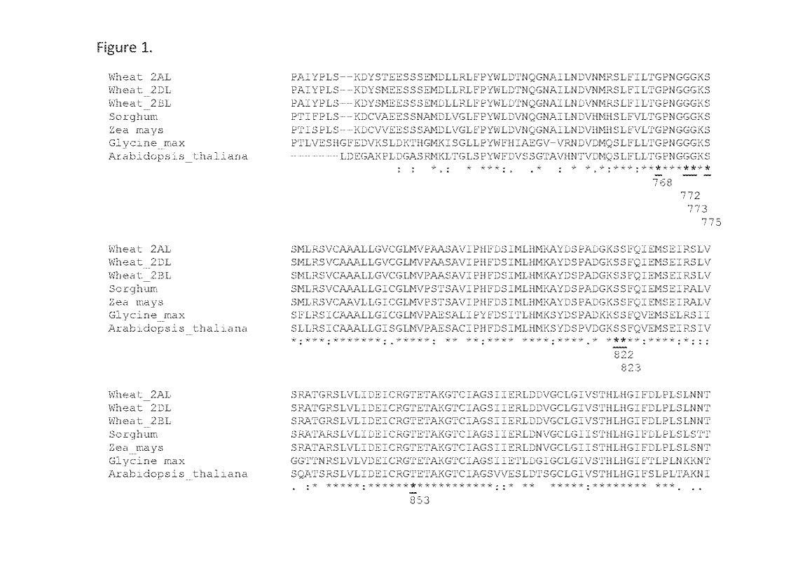

As used herein, the terms “MSH1 equivalent amino acid position” (singular) or “MSH1 equivalent amino acid positions” (plural) refer to amino acid positions 768, 772, 773, 775, 882, 823, and 853 when a plant MSH1 protein is aligned to the plant MSH1 proteins shown in FIG. 1, using the numbering shown in FIG. 1. Programs such as BLAST or CLUSTAL OMEGA or other suitable alignment programs can be used for alignment, with homology criteria of at least 50%, 60%, 70%, 80%, 90% or 95% homology. Insertions or deletions of amino acids in the alignments or protein length do not change the position numbering used herein, e.g., the position of the first G in the peptide sequence SEQ ID NO:8 (GPNGGGKS) that corresponds to G768 in FIG. 1 will have MSH1 equivalent amino acid position 768 in all aligned MSH1 proteins regardless of the position number of the amino acids in the protein being compared.

As used herein, the terms “mutagenesis” or “mutagenizing” or “mutagenized” refer to any random or targeted method of changing a gene's sequence including but not limited to chemical mutagens such as alkylating agents and azides, radiation such as gamma rays, x-rays, and particle radiation such as fast and thermal neutrons, beta and alpha particles as well as gene-editing methods including but not limited to methods such as CRISPR, TALENS, homologous recombination with foreign nucleic acids, and chimeric oligonucleotides.

As used herein, “a plant MSH1 sequence” refers to a mRNA or cDNA or protein or gene sequence of an endogenous MSH1 gene found in a plant.

As used herein, the term “homology” or “homologous” refers to similar protein sequences when aligned by BLAST or CLUSTAL OMEGA or similar alignment methods. Sequences displaying homology or homologous sequences are 70% to 80%, at least 80% to 90%, or 90% to 95%, or 95% to 100% homologous when scored by amino acids at individual positions in the protein that are identical or conservative amino acid substitutions.

As used herein, “conservative amino acid substitutions” are those within the same group: aliphatic (Glycine, Alanine, Valine, Leucine, Isoleucine); Hydroxyl or Sulfur/Selenium-containing (Serine, Cysteine, Selenocysteine, Threonine, Methionine); Cyclic (Proline); Aromatic (Phenylalanine, Tyrosine, Tryptophan); Basic (Histidine, Lysine, Arginine); or Acidic and their Amides (Aspartate, Glutamate, Asparagine, Glutamine).

As used herein, the term “comprising” means “including but not limited to”.

As used herein, the term “progeny” refers to any one of a first, second, third, or subsequent generation obtained from a parent plant or plant derived from a plant cell in culture.

The phrase “operably linked” as used herein refers to the joining of nucleic acid sequences such that one sequence can provide a required function to a linked sequence. In the context of a promoter, “operably linked” means that the promoter is connected to a sequence of interest such that the transcription of that sequence of interest is controlled and regulated by that promoter. When the sequence of interest encodes a protein and when expression of that protein is desired, “operably linked” means that the promoter is linked to the sequence in such a way that the resulting transcript will be efficiently translated. If the linkage of the promoter to the coding sequence is a transcriptional fusion and expression of the encoded protein is desired, the linkage is made so that the first translational initiation codon in the resulting transcript is the initiation codon of the coding sequence. Alternatively, if the linkage of the promoter to the coding sequence is a translational fusion and expression of the encoded protein is desired, the linkage is made so that the first translational initiation codon contained in the 5′ untranslated sequence associated with the promoter is linked such that the resulting translation product is in frame with the translational open reading frame that encodes the protein desired. Nucleic acid sequences that can be operably linked include, but are not limited to, sequences that provide gene expression functions (i.e., gene expression elements such as promoters, 5′ untranslated regions, introns, protein coding regions, 3′ untranslated regions, polyadenylation sites, and/or transcriptional terminators), sequences that provide DNA transfer and/or integration functions (i.e., site specific recombinase recognition sites, integrase recognition sites), sequences that provide for selective functions (i.e., antibiotic resistance markers, biosynthetic genes), sequences that provide scoreable marker functions (i.e., reporter genes), sequences that facilitate in vitro or in vivo manipulations of the sequences (i.e., polylinker sequences, site specific recombination sequences, homologous recombination sequences), and sequences that provide replication functions (i.e., bacterial origins of replication, autonomous replication sequences, centromeric sequences).

As used herein, the term “transgene” or “transgenic” refers to any DNA from in vitro or recombinant DNA source that has been integrated into a chromosome that is stably maintained in a host cell. In this context, sources for the DNA include, but are not limited to, DNAs from an organism distinct from the host cell organism, species distinct from the host cell species, varieties of the same species that are either distinct varieties or identical varieties, DNA that has been subjected to any in vitro modification, recombinant DNA, and any combination thereof.

MSH1 Proteins

Representative MSH1 protein sequences from a non-limiting selection of plants species are provided in Table 1. The Genbank protein accession numbers or protein sequences or SEQ ID numbers are provided. Clustal omega alignment (or a similar alignment program such as BLAST or a program for aligning two or more sequences) of two or more of these proteins with candidate MSH1 proteins is useful for validating a candidate MSH1 protein is an authentic MSH1 protein as authentic MSH1 proteins will be highly conserved with the MSH1 proteins in Table1, especially for plants in similar plant genus, families, order, class or groups. Sequences in Table 1 may not be full length and may not include all the MSH1 family members in polyploid species.

TABLE 1
MSH1 protein sequences from a non-
limiting selection of plant species

| | NCBI Genbank Protein |
|---|---|
| Species | Accession Number |
| Capsella rubella | XP_006299281.1 |
| Ricinus communis | XP_002528340.1 |
| Populus trichocarpa | XP_002314510.1 |
| Theobroma cacao | XP_007035297.1 |
| Citrus sinensis | XP_006480235.1 |
| Citrus clementine | XP_006420379.1 |
| Vitis vinifera | XP_002282256.1 |
| Prunus persica | XP_007225427.1 |
| Fragaria vesca subsp. Vesca | XP_004297941.1 |
| Solanum tuberosum | XP_006340883.1 |
| Cucumis sativus | XP_004134396.1 |
| Solanum lycopersicum | XP_0004247788.1 |
| Cicer arietinum | XP_004497789.1 |
| Glycine max | NP_001238217.1 |
| Phaseolus vulgaris | AAX31514.1 |
| Oryza sativa Japonica Group | NP_001053261.1 |
| Oryza sativa Indica Group | CAH67334.1 |
| Oryza brachyantha | XP_006652491.1 |
| Setaria italic | XP_004976164.1 |
| Medicago truncatula | XP_003590183.1 |
| Zea mays | AFW58800.1 |
| Cucumis sativus | ACA35268.1 |
| Hordeum vulgare subsp. Vulgare | BAK01143.1 |
| Sorghum bicolor | XP_002448138.1 |
| Wheat | SEQ ID NO: 1, SEQ ID |
| | NO: 2, SEQ ID NO: 3 |

Examples of suitable plants for the present invention may include, for example, species of the Family Gramineae, including Sorghum bicolor and Zea mays; species of the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, and Triticum.

In some embodiments, plants or plant cells may include, for example, those from corn (Zea mays), canola (Brassica napes, Brassica raga ssp.), Brassica species useful as sources of seed oil, alfalfa (Medicago sativa), rice (Oryza sativa), rye (Secale cereale), sorghum (Sorghum bicolor, Sorghum vulgate), millet (e.g., pearl millet (Pennisetum glaucum), proso millet (Panicum miliaceum), foxtail millet (Setaria italica), finger millet (Eleusine coracana)), sunflower (Helianthus animus), safflower (Carthamus tinctorius), wheat (Triticum aestivum), duckweed (Lemna), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucijra), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia spp.), almond (Prunus amygdalus), sugar beets (Beta vulgaris), sugarcane (Saccharum spp.), oats, barley, vegetables, ornamentals, and conifers.

Examples of suitable vegetables plants may include, for example, tomatoes (Lycopersicon esculentum), lettuce (e.g., Lactuca sativa), green beans (Phaseolus vulgaris), lima beans (Phaseolus limensis), peas (Lathyrus spp), and members of the genus Cucumis such as cucumber (C. sativus), cantaloupe (C. cantalupensis), and musk melon (C. melo).

Examples of suitable ornamental plants may include, for example, azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Examples of suitable ornamental plants may include, for example, azalea (Rhododendron spp.), hydrangea (Macrophylla hydrangea), hibiscus (Hibiscus rosasanensis), roses (Rosa spp), tulips (Tulipa spp.), daffodils (Narcissus spp.), petunias (Petunia hybrida), carnation (Dianthus caryophyllus), poinsettia (Euphorbia pulcherrima), and chrysanthemum.

Examples of suitable leguminous plants may include, for example, guar, locust bean, fenugreek, soybean, garden beans, cowpea, mungbean, lima bean, fava bean, lentils, chickpea, peanuts (Arachis sp.), crown vetch (Vicia sp.), hairy vetch, adzuki bean, lupine (Lupines sp.), trifolium, common bean (Phaseolus sp.), field bean (Pisum sp.), clover (Melilotus sp) Lotus, trefoil, lens, and false indigo.

Examples of suitable forage and turf grass may include, for example, alfalfa (Medicago s sp.), orchard grass, tall fescue, perennial ryegrass, creeping bent grass, and redtop.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Identification of Candidate MSH1-DN Mutations

Dominant negative mutations previously identified in non-plant species were mapped by protein homology to the Arabidopsis MSH1 protein. A subset of these were selected as candidate MSH1-DN mutations and are identified in Table 2 and their positions within the ATPase domain of selected plant MSH1 proteins is shown in FIG. 1. The MSH1 protein sequences are highly conserved in plants as shown in FIG. 1 and the corresponding position of the candidate MSH1-DN amino acids in other plant MSH1 proteins can be readily identified by alignment as shown in FIG. 1. These candidate MSH1-DN amino acid positions are referred to as MSH1 equivalent amino acid positions and refer to amino acid positions 768, 772, 773, 775, 882, 823, and 853 when a plant MSH1 protein is aligned to the plant MSH1 proteins shown in FIG. 1, using the numbering shown in FIG. 1. Programs such as BLAST or CLUSTAL OMEGA or other suitable alignment programs can be used for alignment, with homology criteria of at least 50%, 60%, 70%, 80%, 90% or 95% homology. Insertions or deletions of amino acids in the alignments or protein length do not change the position numbering used herein, e.g., the position of the first G in the peptide sequence SEQ ID NO:8 GPNGGGKS that corresponds to G768 in FIG. 1 will have MSH1 equivalent amino acid position 768 in all aligned MSH1 proteins regardless of the position number of the amino acids in the protein being compared

### Example 2

### Production of Transgenic Plants Containing MSH1-DN Mutations

A binary vector capable of Agrobacterium-mediated DNA transfer and containing an Arabidopsis Msh1 gene was mutated in the ATP binding domain to contain the amino acid mutations and corresponding DNA mutations are shown in Table 2. Construction of this vector and mutants was accomplished as follows, The base vector is a right border pVS1 binary plasmid capable of DNA transfer into plant cells. This binary vector contains a spectinomycin resistance gene for bacterial selection. For visual selection of transformed seeds a DsRED marker was also present in this vector. This DsRED gene uses a constitutive soybean ubiquitin promoter similar to that reported (Hernandez-Garcia et al. 2010) and a 3′ polyadenylation region from the Cauliflower Mosaic Virus 35S.

An Arabidopsis Msh1 full-length genomic DNA (gDNA), containing and expressed from its native promoter (Virdi et al., 2016, Molecular Plant, 9: 245-260) was sub-cloned at a location 3′ to the DsRED gene in the pVS1 binary vector using standard restriction enzyme digestion and ligation cloning methods. Al the 3′ end of the MSH1 genomic clone, a Cauliflower Mosaic Virus 35S 3′ polyadenylation region is used as the 3′ polyadenylation region was used instead of the native Arabidopsis Msh1 3′ polyadenylation region. To facilitate the cloning of the single-amino acid mutations into the Arabidopsis ATPase domain coding region, two unique restriction sites (XhoI and AvrII) were engineered via silent mutations at positions 5037 nt and 5512 nt, with the numbering respective to the ATG initiation codon in the MSH1 full-length gDNA.

Nine amino acids mutations in Table 2 were targeted as candidate dominant negative mutations. Nine DNA fragments, each encoding one of the amino acid mutations in Table 2, were constructed as follows. First, two PCR fragments, one spanning the region between the XhoI and the mutation region and the second spanning the mutation region to the AvrII restriction sites were amplified using oligonucleotides containing the mutation sequence modification desired. The first PCR fragment was flanked by XhoI and BsaI restriction sites, while the second PCR fragment was flanked by BsaI and AvrII restriction sites. Both PCR amplicons were purified, digested with the corresponding enzymes, and ligated into the base vector previously digested with XhoI and AvrII. The BsaI restriction sites were designed to cleave the DNA adjacent to the BsaI recognition sequence, facilitating the joining of both PCR fragments into the base vector while removing the BsaI recognition sites. The sequence of each of the resulting constructs containing a MSH1 full-length gDNA containing the targeted mutation was verified by DNA sequencing.

The final binary constructs were electroporated into Agrobacterium EHA105 and the resulting Agrobacterium strain were used for floral dip transformation of wild-type Arabidopsis flowering plants. Transgenic seeds are identified by their fluorescent red color.

TABLE 2
Dominant Negative MSH1 candidate amino acid mutations

DNMut1: G768D
SEQ ID NO: 10 TGPNGGGKS → SEQ ID NO: 12 TDPNGGGKS
SEQ ID NO: 9 ACTGGACCTAACGGTGGTGGTAAATCG → SEQ ID NO: 11
ACTGACCCTAACGGTGGTGGTAAATCG
DNMut2: G772S
SEQ ID NO: 10 TGPNGGGKS → SEQ ID NO: 14 TGPNGSGKS
SEQ ID NO: 9 ACFGGACCTAACGGTGGTGGTAAATCG → SEQ ID NO: 13
ACTGGACCTAACGGTTCCGGTAAATCG
DNMut3: G773D
SEQ ID NO: 10 TGPNGGGKS → SEQ ID NO: 16 TGPNGGDKS
SEQ ID NO: 9 ACTGGACCTAACGGTGGTGGTAAATCG → SEQ ID NO: 15
ACTTGGACCTAACGGTGGTGACAAATCG
DNMut4: S775N
SEQ ID NO: 10 TGPNGGGKS → SEQ ID NO: 18 TGPNGGGKN
SEQ ID NO: 9 ACTGGACCTAACGGTGGTGGTAAATCG → SEQ ID NO: 17
ACTGGACCTAACGGTGGTGGTAAAAAC
DNMut5: S822P
SEQ ID NO: 26 KSSFQVE → SEQ ID NO: 20 KPSFQVE
SEQ ID NO: 25 AAAAGTTCTTTCCAGGTAGAA → SEQ ID NO: 19
AAACCTTCTTTCCAGGTAGAA
DNMut6: S822L
SEQ ID NO: 26 KSSFQVE → SEQ ID NO: 22 KLSFQVE
SEQ ID NO: 25 AAAAGTTCYFFCCAGGTAGAA → SEQ ID NO: 21
AAACTTTCTTTCCAGGTAGAA
DMMut7: S823I
SEQ ID NO: 26 KSSFQVE → SEQ ID NO: 24 KSIFQVE
SEQ ID NO: 25 AAAAGTTCTTTCCAGGTAGAA → SEQ ID NO: 23
AAAAGTATCTTCCAGGTAGAA
DNMut8: T853D
SEQ ID NO: 28 GTETAKG → SEQ ID NO: 30 GDETAKG
SEQ ID NO: 27 GGGACAGAGACAGCAAAAGGC → SEQ ID NO: 29
GGGGATGAGACAGCAAAAGGC
DNMut9: T853I
SEQ ID NO: 28 GTETAKG → SEQ ID NO: 32 GIETAKG
SEQ ID NO: 27 GGGACAGAGACAGCAAAAGGC → SEQ ID NO: 31
GGGATTGAGACAGCAAAAGGC

Mutations are numbered DNMut1 to DNMut9 and show the amino acid change flanking the position number. The position numbers are shown in FIG. 1. Below each mutant name is the amino acid motif with the wild type sequence to the left of the arrow and the DN mutation to the right of the arrow. Below these are the corresponding cDNA sequences of the normal and DN mutation genes. The amino acid and codon changes are underlined and in bold.

### Example 3

### Screening Arabidopsis Plants for MSH1-DN Mutations that Inhibit Wild Type MSH1

Transgenic plants were grown from DsRED positive T1 seeds and examined for light green, yellow, or white leaf variegation, leaf morphology changes, and flowering time, as functional indicators of impaired MSH1 function. T1 plants were grown to maturity, seeds collected, and DsRED T2 seeds identified. These T2 plants were also examined for light green, yellow, or white leaf variegation, leaf morphology changes, and flowering time, indicative of impaired MSH1 function as loss of MSH1 function often has stronger variegation phenotypes in the second generation for loss of MSH1 function. Lineages showing leaf variegation in the T1 and/or T2 generation identified MSH1-DN mutations capable of suppressing the function of the endogenous wild type MSH1 protein as these plants have two copies of wild type endogenous Msh1 genes in addition to the MSH1-DN mutated transgene(s). A number of independently transformed transgenic T1 and T2 progeny from dominant negative mutants G772S and S823I displayed loss of MSH1 function phenotypes, indicating these were the most efficient dominant negative mutants for inhibiting wild type MSH1 function.

### Example 4

### CRISPR Gene Editing to Introduce MSH1-DN Mutations into Endogenous Msh1 Genes

CRISPR gene editing technologies are used for targeted mutagenesis at plant genes targeted by suitably designed guide RNAs against the ATPase domain of endogenous MSH1 genes. The breakage-repair reactions following CRISPR cleavage creates useful mutations that are screened by PCR/DNA sequencing or Taqman SNP detection methods (or equivalent SNP methods) for the desired MSH1-DN mutations in cells or plants. Introduction of a DNA repair template and a Msh1-Targeted CRISPR/guide RNA increases the frequency of recovering targeted MSH1-DN mutations (for a recent review of CRISPR/CAS9 in plants, see Bortesi and Fischer, 2015, Biotechnology Advances 33(10): 41-52).

### Example 5

### EMS Mutagenesis to Produce MSH1-DN Mutations in Plant Endogenous Genes

EMS methods of creating random point mutations are well known with published protocols for treating soybean seeds or maize pollen. Once mutagenized M2 seeds have been produced from the progeny of the mutagenized plants, pooling of the seeds and next generation sequencing of pools of DNA from the pooled seeds is used to identify M2 plants with the desired sequence change. A plant containing a MSH1-DN mutation in an endogenous Msh1 gene is produced by this EMS/screening method.

### REFERENCES

Hernandez-Garcia, C M, Bouchard, R, Rushton, P, Jones, M, Chen, X, Timko, M, and Finer, J (2010) High level transgenic expression of soybean (Glycine max) GmERF and Gmubi gene promoters isolated by a novel promoter analysis pipeline. BMC Plant Biol. 10: 237.

Virdi, K S, Wamboldt, Y, Kundariya, H, Laurie, J D, Keren, I, Sunil Kumar, K R, Block, A, Basset, G. Luebker, S, Elowsky. C. Day, P M, Roose, J L, Bricker, T M, Elthon, T, and Mackenzie, S A (2016) MSH1 Is a Plant Organellar DNA Binding and Thylakoid Protein under Precise Spatial Regulation to Alter Development. Molecular Plant. 9: 245-260.

What is claimed is:

1. A method of identifying a MSH1-DN mutation comprising:
a. Producing candidate MSH1-DN mutations in an isolated Msh1 gene that is subsequently transformed into a plant;
b. Screening plants or their progeny comprising said candidate MSH1-DN mutations of step (a) for loss of MSH1 function when containing both mutant and normal Msh1 genes; and
c. Identifying a MSH1-DN mutation that suppresses MSH1 function in a plant containing both mutant and normal Msh1 genes.

2. The method of claim 1, herein a MSH1-DN mutation in a endogenous Msh1 gene in a plant obtained by:
a. Mutagenizing a population of plant cells, seeds, or pollen, and producing plants from the mutagenized plant cells, seeds, or pollen;
b. Screening said plants of step (a), or their progeny, to identify plants comprising a MSH1-DN mutation identified by the method of claim 1; and
c. Identifying a plant comprising a MSH1-DN mutation.

3. The method of claim 1, wherein a MSH1-DN mutation in a endogenous Msh1 gene in a plant is obtained by:
a. Mutagenizing a population of plant cells, seeds, or pollen, and producing plants from the mutagenized plant cells, seeds, or pollen;
b. Screening said plants of step (a), or their progeny, to identify plants comprising a MSH1-DN mutation at an MSH1 equivalent amino acid position selected from the group consisting of positions of the MSH1-DN mutations in Table 2; and
c. Identifying a plant comprising a MSH1-DN mutation.

4. The method of claim 3, wherein the MSH1-DN mutation is selected from the group of MSH1-DN mutations G772S and S823I.

5. A plant or crop plant comprising a MSH1-DN mutation at an MSH1 equivalent amino acid position selected from the group consisting of positions of the MSH1-DN mutations in Table 2.

6. A plant or crop plant of claim 5, comprising a MSH1-DN mutation is selected from the group of MSH1-DN mutations G772S and S823I.

7. A method for producing a plant exhibiting improved yield comprising the steps of:
a. suppressing expression of endogenous MSH1 gene(s) in a plant or plant cell to obtain a first parental plant, wherein suppression of MSH1 expression is effected with a MSH1-DN;
b. selfing the first parental plant or crossing the first parental plant to a second parental plant;
c. recovering progeny plants or a progeny plant line from the self or cross of the first parental plant of step (b) wherein MSH1 function is restored; and,
d. selecting a progeny plant or a progeny plant line from a recovered progeny plant or plant line of step (c) or from a self or outcross of a recovered progeny plant or plant line of step (c) wherein the selected progeny plant or plant line exhibits improved yield in comparison to a control plant, wherein said improved yield is associated with one or more epigenetic changes in the nucleus of the progeny plant cells relative to the corresponding parental chromosomal loci and is heritable.

8. The method of claim 7, wherein said MSH1-DN mutation of step (a) is at an MSH1 equivalent amino acid position selected from the group consisting of non-naturally occurring amino acids at MSH1 equivalent amino acid positions 768, 772, 773, 775, 882, 823, and 853.

9. The method of claim 8, wherein said MSH1-DN mutation is at MSH1 equivalent amino acid position and is selected from the group of MSH1-DN mutations in Table 2.


SEQUENCE LISTING (32 sequences)

SEQ ID NO: 1 - protein, 178 aa, Triticum aestivum
  ACT_SITE (1)..(178): ATP binding domain
PAIYPLSKDYSTEESSSEMDLLRLFPYWLDTNQGNAILNDVNMRSLFILTGPNGGGKSSMLRSVCAAALLGVCGLMVPAASAVIPHFDSIMLHMKAYDSPADGKSSFQIEMSEIRSLVSRATGRSLVLIDEICRGTETAKGTCIAGSIIERLDDVGCLGIVSTHLHGIFDLPLSLNNT

SEQ ID NO: 2 - protein, 178 aa, Triticum aestivum
  ACT_SITE (1)..(178): ATP binding domain
PAIYPLSKDYSMEESSSEMDLLRLFPYWLDTNQGNAILNDVNMRSLFILTGPNGGGKSSMLRSVCAAALLGVCGLMVPAASAVIPHFDSIMLHMKAYDSPADGKSSFQIEMSEIRSLVSRATGRSLVLIDEICRGTETAKGTCIAGSIIERLDDVGCLGIVSTHLHGIFDLPLSLNNT

SEQ ID NO: 3 - protein, 178 aa, Triticum aestivum; identical to SEQ ID NO: 2
  ACT_SITE (1)..(178)

SEQ ID NO: 4 - protein, 178 aa, Sorghum bicolor
  ACT_SITE (1)..(178)
PTIFPLSKDCVAEESSNAMDLVGLFPYWLDVNQGNAILNDVHMHSLFVLTGPNGGGKSSMLRSVCAAALLGICGLMVPSTSAVIPHFDSIMLHMKAYDSPADGKSSFQIEMSEIRALVSRATARSLVLIDEICRGTETAKGTCIAGSIIERLDNVGCLGIISTHLHGIFDLPLSLSTT

SEQ ID NO: 5 - protein, 178 aa, Zea mays
  ACT_SITE (1)..(178)
PTISPLSKDCVVEESSSAMDLVGLFPYWLDVNQGNAILNDVHMHSLFVLTGPNGGGKSSMLRSVCAAVLLGICGLMVPSTSAVIPHFDSIMLHMKAYDSPADGKSSFQIEMSEIRALVSRATARSLVLIDEICRGTETAKGTCIAGSIIERLDNVGCLGIISTHLHGIFDLPLSLSNT

SEQ ID NO: 6 - protein, 179 aa, Glycine_max
  ACT_SITE (1)..(179)
PTLVESHGFEDVKSLDKTHGMKISGLLPYWFHIAEGVVRNDVDMQSLFLLTGPNGGGKSSFLRSICAAALLGICGLMVPAESALIPYFDSITLHMKSYDSPADKKSSFQVEMSELRSIIGGTTNRSLVLVDEICRGTETAKGTCIAGSIIETLDGIGCLGIVSTHLHGIFTLPLNKKNT

SEQ ID NO: 7 - protein, 173 aa, Arabidopsis_thaliana
  ACT_SITE (1)..(173)
LDEGAKPLDGASRMKLTGLSPYWFDVSSGTAVHNTVDMQSLFLLTGPNGGGKSSLLRSICAAALLGISGLMVPAESACIPHFDSIMLHMKSYDSPVDGKSSFQVEMSEIRSIVSQATSRSLVLIDEICRGTETAKGTCIAGSVVESLDTSGCLGIVSTHLHGIFSLPLTAKNI

SEQ ID NO: 8 - protein, 8 aa, Arabidopsis thaliana
  ACT_SITE (1)..(8)
GPNGGGKS

SEQ ID NO: 9 - DNA, 27 bp, Arabidopsis thaliana
  CDS (1)..(27)
actggacctaacggtggtggtaaatcg

SEQ ID NO: 10 - protein, 9 aa, Arabidopsis thaliana
TGPNGGGKS

SEQ ID NO: 11 - DNA, 27 bp, Arabidopsis thaliana
  CDS (1)..(27)
actgaccctaacggtggtggtaaatcg

SEQ ID NO: 12 - protein, 9 aa, Arabidopsis thaliana
TDPNGGGKS

SEQ ID NO: 13 - DNA, 27 bp, Arabidopsis thaliana
  CDS (1)..(27)
actggacctaacggttccggtaaatcg

SEQ ID NO: 14 - protein, 9 aa, Arabidopsis thaliana
TGPNGSGKS

SEQ ID NO: 15 - DNA, 27 bp, Arabidopsis thaliana
  CDS (1)..(27)
actggacctaacggtggtgacaaatcg

SEQ ID NO: 16 - protein, 9 aa, Arabidopsis thaliana
TGPNGGDKS

SEQ ID NO: 17 - DNA, 27 bp, Arabidopsis thaliana
  CDS (1)..(27)
actggacctaacggtggtggtaaaaac

SEQ ID NO: 18 - protein, 9 aa, Arabidopsis thaliana
TGPNGGGKN

SEQ ID NO: 19 - DNA, 21 bp, Arabidopsis thaliana
  CDS (1)..(21)
aaaccttctttccaggtagaa

SEQ ID NO: 20 - protein, 7 aa, Arabidopsis thaliana
KPSFQVE

SEQ ID NO: 21 - DNA, 21 bp, Arabidopsis thaliana
  CDS (1)..(21)
aaactttctttccaggtagaa

SEQ ID NO: 22 - protein, 7 aa, Arabidopsis thaliana
KLSFQVE

SEQ ID NO: 23 - DNA, 21 bp, Arabidopsis thaliana
  CDS (1)..(21)
aaaagtatcttccaggtagaa

SEQ ID NO: 24 - protein, 7 aa, Arabidopsis thaliana
KSIFQVE

SEQ ID NO: 25 - DNA, 21 bp, Arabidopsis thaliana
  CDS (1)..(21)
aaaagttctttccaggtagaa

SEQ ID NO: 26 - protein, 7 aa, Arabidopsis thaliana
KSSFQVE

SEQ ID NO: 27 - DNA, 21 bp, Arabidopsis thaliana
  CDS (1)..(21)
gggacagagacagcaaaaggc

SEQ ID NO: 28 - protein, 7 aa, Arabidopsis thaliana
GTETAKG

SEQ ID NO: 29 - DNA, 21 bp, Arabidopsis thaliana
  CDS (1)..(21)
ggggatgagacagcaaaaggc

SEQ ID NO: 30 - protein, 7 aa, Arabidopsis thaliana
GDETAKG

SEQ ID NO: 31 - DNA, 21 bp, Arabidopsis thaliana
  CDS (1)..(21)
gggattgagacagcaaaaggc

SEQ ID NO: 32 - protein, 7 aa, Arabidopsis thaliana
GIETAKG